(19) United States
(12) Patent Application Publication
(10) Pub. No.: US 2019/0320774 A1
(43) Pub. Date: Oct. 24, 2019

(54) HAIR COLORING MEMBRANE ASSEMBLY

(71) Applicants: Laurie LaRoche, Singer Island, FL (US); George Ryan, Singer Island, FL (US); Russell Gnann, Singer Island, FL (US)

(72) Inventors: Laurie LaRoche, Singer Island, FL (US); George Ryan, Singer Island, FL (US); Russell Gnann, Singer Island, FL (US)

(21) Appl. No.: 15/957,066

(22) Filed: Apr. 19, 2018

(51) Int. Cl. A45D 19/00; B65H 16/00; A61Q 5/10; A61K 8/02

(52) U.S. Cl. CPC A45D 19/0016; B65H 16/005; A61Q 5/10; A61K 2800/43; A45D 2019/0083; A45D 2200/1036; A61K 2800/87; A61K 8/0216

(57) ABSTRACT

A hair coloring membrane assembly for enhancing the dye process of grey hair includes a dispenser that has an opening therein. A plurality of strips is positioned in the dispenser and is selectively removable from the dispenser. Thus, each of the strips may be positioned on a scalp during hair coloring. Each of the strips comprises a first layer, a second layer and a third layer. Each of the first layer and the third layer is comprised of a fluid absorbent material to transfer a fluid hair dye onto the scalp thereby enhancing the fluid hair dye to penetrate roots of hair being dyed. The second layer is comprised of a light reflective material and the second layer is infused with a fluid emulsifier. The fluid emulsifier is released into the hair thereby enhancing absorption of the fluid hair dye into the hair.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to membrane devices and more particularly pertains to a new membrane device for enhancing the dye process for grey hair.

### BRIEF SUMMARY OF THE INVENTION

An embodiment of the disclosure meets the needs presented above by generally comprising a dispenser that has an opening therein. A plurality of strips is positioned in the dispenser and is selectively removable from the dispenser. Thus, each of the strips may be positioned on a scalp during hair coloring. Each of the strips comprises a first layer, a second layer and a third layer. Each of the first layer and the third layer is comprised of a fluid absorbent material to transfer a fluid hair dye onto the scalp thereby enhancing the fluid hair dye to penetrate roots of hair being dyed. The second layer is comprised of a light reflective material and the second layer is infused with a fluid emulsifier. The fluid emulsifier is released into the hair thereby enhancing absorption of the fluid hair dye into the hair.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a hair coloring membrane assembly according to an embodiment of the disclosure.

FIG. 2 is a right side phantom view of an embodiment of the disclosure.

FIG. 3 is a top view of an embodiment of the disclosure.

FIG. 4 is a cross sectional view taken along line 4-4 of FIG. 3 of an embodiment of the disclosure.

FIG. 5 is a perspective in-use view of an embodiment of the disclosure.

### DETAILED DESCRIPTION OF THE INVENTION

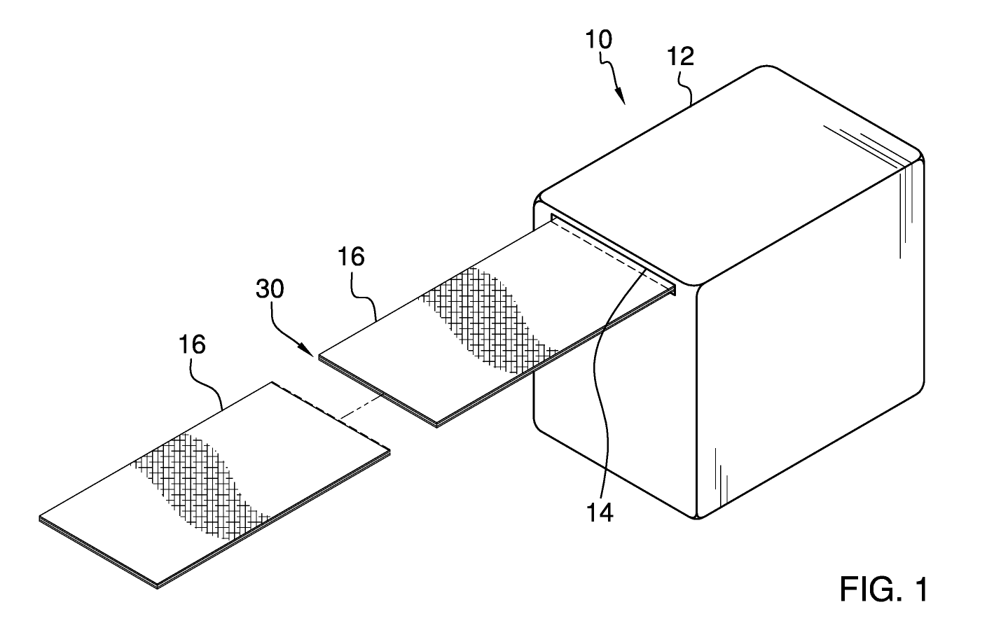

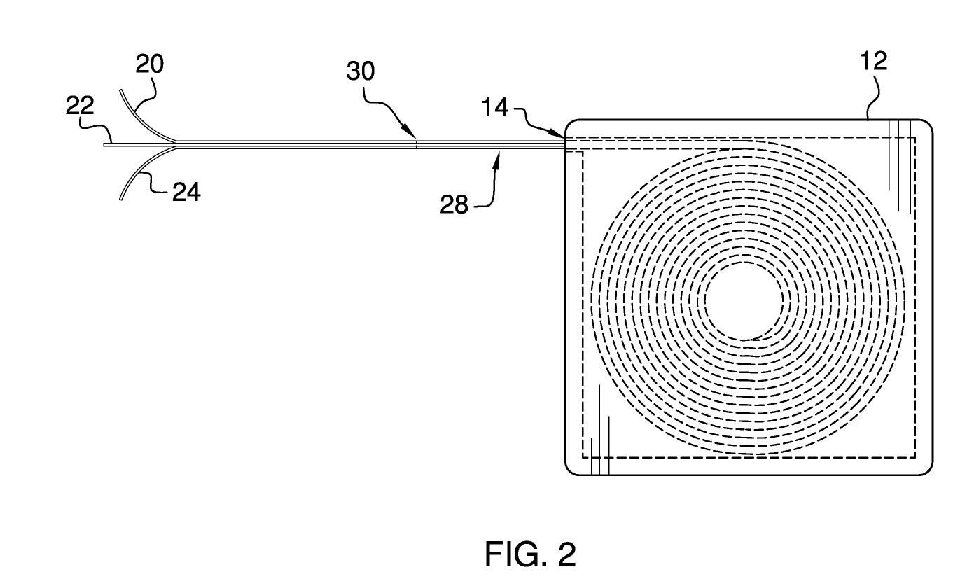

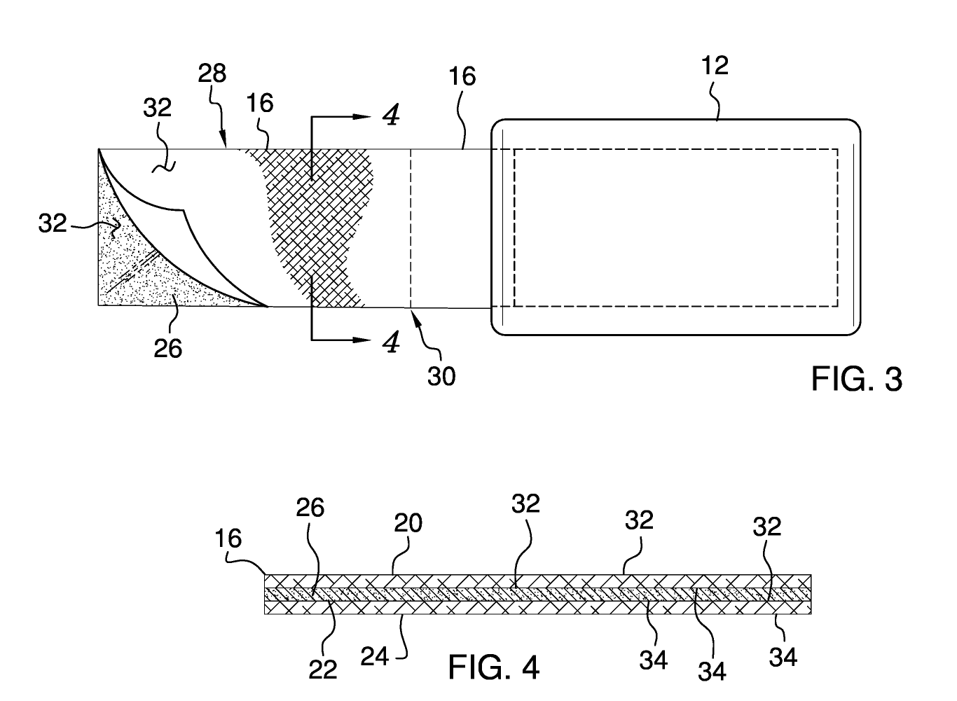

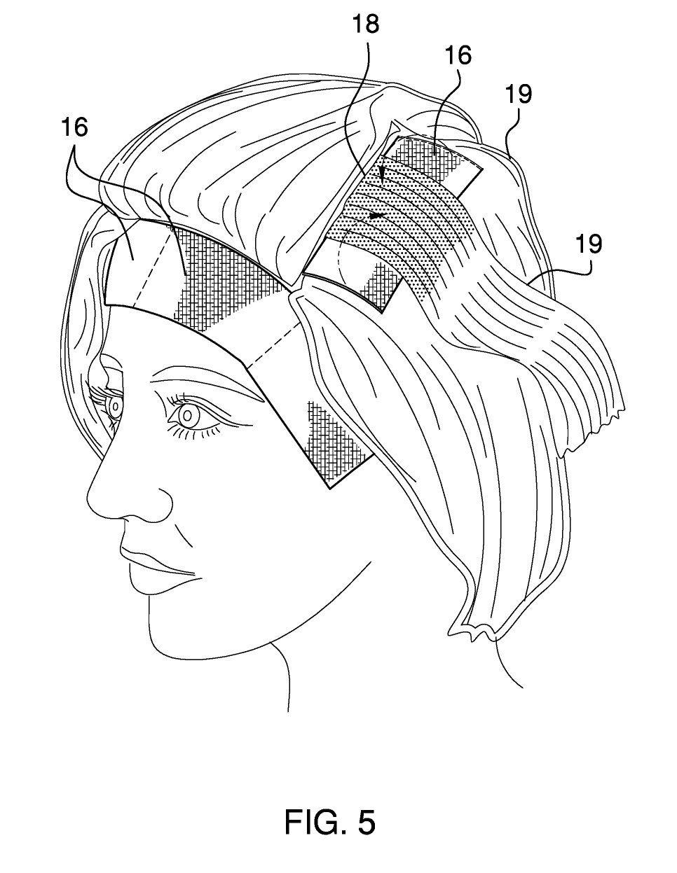

With reference now to the drawings, and in particular to FIGS. 1 through 5 thereof, a new membrane device embodying the principles and concepts of an embodiment of the disclosure and generally designated by the reference numeral 10 will be described.

As best illustrated in FIGS. 1 through 5, the hair coloring membrane assembly 10 generally comprises a dispenser 12 that has an opening 14 therein and the dispenser 12 is hollow. The dispenser 12 may have a cubic shape or any other shape. Alternatively, the dispenser 12 may have a stem for receiving a spool and a cutting blade in the convention of a Scotch Tape dispenser or the like. A plurality of strips 16 is provided and the plurality of strips 16 is positioned in the dispenser 12 and is selectively removable from the dispenser 12. Each of the strips 16 is positionable on a scalp 18 during hair coloring and each of the strips 16 is wrapped around strands of hair 19 on the scalp 18.

Each of the strips 16 comprises a first layer 20, a second layer 22 and a third layer 24. The first layer 20 and the third layer 24 of each strip are comprised of a fluid absorbent material. In this way the first layer 20 and the third layer 24 of each strip may absorb and thusly transfer a fluid hair dye onto the scalp 18 and the base of the strands of hair 19 around which each strip 16 is wrapped when hair 19 on the scalp 18 is being dyed for cosmetic purposes. Thus, the strips 16 enhance the fluid hair dye penetrating roots of hair 19 that is being dyed.

The second layer 22 is comprised of a foil like, light reflective material. Moreover, the second layer 22 is infused with a fluid emulsifier 26 such as coconut oil, keratin or other emulsifier that is non toxic to human skin and hair. The second layer 22 releases the fluid emulsifier 26 into the hair 19 when the strips 16 are positioned on the scalp 18 and wrapped around the strands of hair 19. In this way the fluid emulsifier 26 enhances absorption of the fluid hair dye into the hair 19. Additionally, the fluid emulsifier 26 specifically enhances softness, texture and absorption of grey hair 19 thereby enhancing the appearance of grey hair 19 when the grey hair 19 is dyed.

The plurality of strips 16 is releasably coupled together and are oriented co-linear with each other such that the plurality of strips 16 defines an elongated band 28. The elongated band 28 is rolled upon itself and is positioned within the dispenser 12. Additionally, the elongated band 28 extends outwardly through the opening 14 in the dispenser 12. Thus, each of the strips 16 is selectively releasable from the elongated band 28. The elongated band 28 may have a plurality of perforations 30 that are spaced apart from each other to define each of the strips 16. Moreover, the perforations 30 may be torn to release each of the strips 16 from the elongated band 28.

Each of the first layer 20, the second layer 22 and the third layer 24 has a first surface 32 and a second surface 34. The second surface 34 of the first layer 20 is bonded to the first surface 32 of the second layer 22 having the first surface 32 of the first layer 20 being exposed. The first surface 32 of the third layer 24 is bonded to the second surface 34 of the second layer 22 having the second surface 34 of the third layer 24 being exposed. The second surface 34 of the third layer 24 rests on the scalp 18 when the strips 16 are positioned on the scalp 18.

In use, a selected number of strips 16 are removed from the elongated band 28 and each of the selected number of strips 16 is positioned at selected spots on the scalp 18 when the hair 19 is being dyed. Each of the strips 16 is wrapped around strands of hair 19 that are adjacent to each of the strips 16. Each of the first layer 20 and the second layer 22 absorbs the fluid hair dye when the fluid hair dye is applied to the hair 19. In this way the first layer 20 and the second layer 22 transfer the fluid hair dye to a bottom of each hair 19 shaft and onto the root of each hair 19 shaft. In this way the strips 16 enhance the fluid hair dye to be absorbed into a full length of each hair 19 thereby enhancing the appearance of the hair 19 when the hair 19 is finished being dyed. The second layer 22 releases the fluid emulsifier 26 onto the hair 19 when the hair 19 is being dyed thereby increasing the absorption rate of each hair 19 with respect to the fluid hair dye and to enhance softness and texture of each hair 19. Thus, each of the strips 16 increases the absorption of the fluid hair dye into each hair 19 and each of the strips 16 enhances the overall health of the hair 19.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A hair coloring membrane assembly being configured to be positioned on a scalp during hair coloring for enhancing absorption of a hair dye, said assembly comprising:
a dispenser having an opening therein, said dispenser being hollow; and
a plurality of strips, said plurality of strips being positioned in said dispenser and being selectively removable from said dispenser wherein each of said strips is configured to be positioned on a scalp during hair coloring, each of said strips comprising a first layer, a second layer and a third layer, each of said first layer and said third layer being comprised of a fluid absorbent material wherein each of said first layer and said third layer is configured to transfer a fluid hair dye onto the scalp thereby enhancing the fluid hair dye to penetrate roots of hair being dyed, said second layer being comprised of a light reflective material, said second layer being infused with a fluid emulsifier wherein said fluid emulsifier is configured to be released into the hair thereby enhancing absorption of the fluid hair dye into the hair.

2. The assembly according to claim 1, wherein said plurality of strips is releasably coupled together and being oriented co-linear with each other such that said plurality of strips defines an elongated band.

3. The assembly according to claim 2, wherein said elongated band is rolled upon itself and being positioned within said dispenser, said elongated band extending outwardly through said opening in said dispenser wherein each of said strips is selectively releasable from said elongated band.

4. The assembly according to claim 1, wherein each of said first layer, said second layer and said third layer has a first surface and a second surface.

5. The assembly according to claim 4, wherein said second surface of said first layer is bonded to said first surface of said second layer having said first surface of said first layer being exposed.

6. The assembly according to claim 5, wherein said first surface of said third layer is bonded to said second surface of said second layer having said second surface of said third layer being exposed.

7. A hair coloring membrane assembly being configured to be positioned on a scalp during hair coloring for enhancing absorption of a hair dye, said assembly comprising:
a dispenser having an opening therein, said dispenser being hollow; and
a plurality of strips, said plurality of strips being positioned in said dispenser and being selectively removable from said dispenser wherein each of said strips is configured to be positioned on a scalp during hair coloring, each of said strips comprising a first layer, a second layer and a third layer, each of said first layer and said third layer being comprised of a fluid absorbent material wherein each of said first layer and said third layer is configured to transfer a fluid hair dye onto the scalp thereby enhancing the fluid hair dye to penetrate roots of hair being dyed, said second layer being comprised of a light reflective material, said second layer being infused with a fluid emulsifier wherein said fluid emulsifier is configured to be released into the hair thereby enhancing absorption of the fluid hair dye into the hair, said plurality of strips being releasably coupled together and being oriented co-linear with each other such that said plurality of strips defines an elongated band, said elongated band being rolled upon itself and being positioned within said dispenser, said elongated band extending outwardly through said opening in said dispenser wherein each of said strips is selectively releasable from said elongated band, each of said first layer, said second layer and said third layer having a first surface and a second surface, said second surface of said first layer being bonded to said first surface of said second layer having said first surface of said first layer being exposed, said first surface of said third layer being bonded to said second surface of said second layer having said second surface of said third layer being exposed.
